(19) United States
(12) Patent Application Publication
(10) Pub. No.: US 2017/0224043 A1
(43) Pub. Date: Aug. 10, 2017

(54) HELMET AND GOGGLES FOR USE THEREWITH AND METHOD OF USING THE SAME

(71) Applicant: Kimpex Inc., Drummondville (CA)

(72) Inventors: Nicolas BOUCHARD-FORTIN, Racine (CA); Robert HANDFIELD, St-Lucien (CA); Stephane DION, Sainte-Cecile de Milton (CA)

(21) Appl. No.: 15/425,768

(22) Filed: Feb. 6, 2017

Related U.S. Application Data
(60) Provisional application No. 62/291,723, filed on Feb. 5, 2016.

(51) Int. Cl. A42B 3/32; A61F 9/02; A42B 3/20; A42B 3/18

(52) U.S. Cl. CPC A42B 3/326; A42B 3/185; A61F 9/028; A61F 9/029; A42B 3/205

(57) ABSTRACT

A helmet having a movable and openable jaw shield is disclosed. The movable jaw shield is pivotable about a hinge located on the front of the helmet chin bar. The movable jaw shield in open configuration clears the space in front of the user's mouth to facilitate communications from the user. Preferably, the movable jaw shield may also be detached from the pivot to be removed or interchanged. The movable jaw shield is suited with a breath guard configured to mate with a protrusion on the lower front portion of goggles frames. The mating of the goggles and breath guard preferably substantially prevent environmental foreign materials from entering between the breath guard and goggles.

### CROSS-REFERENCE TO RELATED APPLICATIONS

The present patent application claims the benefits of priority of U.S. Provisional Patent Application No. 62/291,723, entitled “Movable Jaw Shield Helmet and Method of Using the Same”, and filed at the United States Patent and Trademark Office on Feb. 5, 2016.

### FIELD OF THE INVENTION

The present invention generally relates to the field of sport equipment, and more precisely sporting protective devices such as helmets and goggles for use therewith.

### BACKGROUND OF THE INVENTION

The preoccupation of designing protective headgears for a variety of activities has been present for many decades. Over the years, the number of activities requiring this kind of equipment has increased the nature of the dangers associated with these activities. The expectations of the users did also modify over the years.

Accordingly, there is a need for sporting protective helmet that provides the safety users desire as the features they expect, one of them being the presence of a jaw shield as a component of the helmet to protect the mouth of the user from different sources of danger or discomfort.

Examples of development in the field involving a jaw shield or another component to protect the jaw of the user include U.S. Pat. No. 4,869,836 (Vitaloni) which teaches the use of a chin guard that is rotatively mounted on the helmet and rotates downwardly in front of the mouth of the user. When in that position, the chin guard gets in contact with the throat of the user wearing the helmet. U.S. Pat. No. 7,120,940 (Fournier et al.) describes the use of a breath mask adjuster coupled to a detachable portion of the helmet.

None of the prior art in the field succeeded in offering to the users a combination of security features and comfort features the present helmet provides.

### SUMMARY OF THE INVENTION

The shortcomings of the prior art are generally mitigated by the present helmet which provides a solution such that the user of the helmet can easily modify the state of the helmet from a closed configuration wherein the jaw shield protects the mouth of the user to an opened configuration wherein the jaw shield is moved to free the space in front of the mouth of the person wearing the helmet. Therefore, the users can more easily be heard by other persons when they speak.

One aspect of one or more embodiments of the present invention provides an improved helmet that comprises a variety of features that simplify and improve the usage of the helmet.

An additional aspect of one or more embodiments of the present invention provides a helmet having a detachable/movable jaw shield easily controllable using a gloved hand.

A further aspect of one or more embodiments of the present invention provides a helmet that comprises a head portion and a jaw shield. The jaw shield comprises a fixed portion rigidly connected to the head portion and a moveable portion disposed at a lower, front, middle portion of the helmet adjacent to the fixed portion and removably attached thereto. According to a further aspect of one or more of these embodiments, the fixed portion of the jaw shield comprises right and left portions that extend forwardly and inwardly toward each other from right and left sides of the head portion. According to a further aspect of one or more of these embodiments, the helmet also comprises a separable hinge that comprises a first part connected to the moveable portion and a second part connected to the fixed portion, the second part being selectively engageable with the first part.

According to a further aspect of one or more of these embodiments, the moveable portion pivots outwardly and downwardly relative to the fixed portion.

According to a further aspect of one or more of these embodiments, the helmet also comprises latches to hold the detachable portion to the fixed portion when in the attached position. The latches engage the fixed portion when the moveable portion is in the attached position.

According to a further aspect of one or more of these embodiments, one of the first and second parts comprises C-shaped clip extending between free ends of the fixed portion. The other of the first and second parts comprises a pin that is engageable with the C-shaped clip such that the pin and C-shaped section are rotatable relative to each other when engaged.

According to a further aspect of one or more of these embodiments, the latch mechanism comprises at least one hook disposed on one of first and second lateral sides of the moveable portion, and at least one catch plate disposed on one of first and second lateral side edges of the fixed portion. The at least one hook selectively engages the at least one catch plate when the moveable portion is in the attached position.

According to a further aspect of one or more of these embodiments, the moveable portion is completely movable from the fixed portion.

A further aspect of one or more embodiments of the present invention provides a helmet that comprises a head portion and a jaw shield. The jaw shield comprises a left portion having a rear end and a forward end, the left portion connected to the head portion at the rear end of the left portion, a right portion having a rear end and a forward end, the right portion connected to the hear portion at the rear end of the right portion, and a moveable portion disposed adjacent the forward ends of the left and right portions. The movable portion according to an aspect of the present invention has a breath guard integrated thereto.

According to a further aspect of the present invention, the movable portion of the helmet is configured to mate with a lower portion of the helmet goggles.

The helmet accordingly comprises a head portion that is the main component of the helmet, and a jaw shield pivotably coupled to the head portion. The movable jaw shield is coupled to the head portion according to two pivot elements, each one situated on a side of the head portion at the low front edge of the head portion. When the helmet is in a closed configuration, namely the configuration in which the mouth of the user is protected by the jaw shield, the side edges of the jaw shield mate with the edges of the head portion. When the helmet is in an opened configuration, the edges of the jaw shield and of the head portion of the helmet are not touching each other. There are connected through the pivot element. At that position, the jaw shield is in a lower position that the bottom of the head portion of the helmet, freeing the space in front of the mouth of the person wearing the helmet.

The helmet further comprises a robust outer shell (providing protection) an inner layer made of compressible material for comfort and additional security. The jaw shield may also comprise a similar set of an outer shell and an inner layer. So when the helmet is in a closed configuration, the outer shells of the head portion and the jaw shield are substantially mating and the inner layers are compressively mating so that the mating of the two components generates substantially airtight surfaces for maximum comfort.

The pivot element joining the head portion and the jaw shield is according to one realization a joint. With the position of the pivot elements designed to be at a distance in front of the throat of the user wearing the helmet and the center of mass of the jaw shield, the helmet thereby results in the jaw shield hanging in the opened configuration while not touching to the throat of the user.

According to an aspect of the present invention, a movable jaw shield helmet having a breath guard mounted thereto, is configured to create a breath box wherein the breathable air is maintained at the mouth and nose level and prevented from interfering with the goggles acting as visual shield. Such a configuration in a motocross (MX) helmet is desired to improve the user's comfort and security, the latter being at least partly in relation to the decrease presence of fog in the goggles.

In other realizations, mechanical elements are used to control the position of the jaw shield in the opened configuration as means used to keep the helmet in the closed configuration when desired.

In accordance with the present disclosure, the disclosed MX helmet comprises a head portion and a jaw shield, the jaw shield comprising a fixed portion and a movable portion. The fixed portion of the jaw shield being rigidly connected to the head portion and extending forwardly therefrom at least in part beyond the chin of a wearer when wearing the helmet. The moveable portion being disposed adjacent the fixed portion in front of the wearer's nose and mouth when wearing the helmet. The moveable portion generally having a lower portion and an upper portion. The lower portion of the movable portion being removably attached to the fixed portion via, at least in part, a hinge comprising a first part connected to the moveable portion and a second part connected to the fixed portion, the second part being selectively engageable with the first part. The upper portion of the movable portion being in contact with the nose and face of the user. As such, contact between the upper portion and the nose of the user allows improved air control. For instances, the helmet in such a configuration may prevent environmental element such as air, dirt, sand, snow and water from entering between the helmet chin bar (movable shield) and the user's nose and cheeks.

Also in accordance with the principles of the present disclosure, the disclosed helmet assembly comprises goggles and a helmet. The helmet comprises a head portion and a jaw shield. The jaw shield having a fixed portion rigidly connected to the head portion, and extending forwardly therefrom at least in part beyond the chin of a wearer when wearing the helmet and a moveable portion disposed adjacent the fixed portion in front of the wearer's nose and mouth when wearing the helmet, the moveable portion having a lower portion and an upper portion. The lower portion of the movable portion being removably attached to the fixed portion via, at least in part, a hinge comprising a first part connected to the moveable portion and a second part connected to the fixed portion, the second part being selectively engageable with the first part. The upper portion being in contact with the goggles. The upper portion being received by the goggles' protrusion or lower front lip to create a sealed helmet inner (user's head) portion.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is a side view of an helmet in accordance with the principle of the present invention;

FIG. 2 is a front view of the helmet of FIG. 1;

FIG. 3 is a perspective view of the helmet of FIG. 1 having a movable jaw portion in an closed configuration;

FIG. 4 is a perspective view of the helmet of FIG. 1 having a movable jaw portion in an opened configuration;

FIG. 5 is a perspective view of the helmet of FIG. 1 having the movable jaw portion removed;

FIG. 6 is a partial profile view of the movable jaw portion of the helmet in a closed configuration;

FIG. 7 is a partial profile view of the jaw portion of the helmet having the movable jaw portion in opened configuration;

FIG. 8 is a partial profile view of the jaw portion of the helmet having the movable jaw portion removed;

FIG. 9 is a front view of a helmet in accordance with the principle of the present invention having goggles mounted thereto with the movable jaw portion in an opened configuration;

FIG. 10 is a perspective view of the helmet of FIG. 9 with the movable jaw in the opened configuration;

FIG. 11 is a side elevation view of the helmet of FIG. 9 having the jaw in the closed position;

FIG. 12 is a perspective view of goggles in accordance with the present invention;

FIG. 13 is a top view of the goggles of FIG. 12;

FIG. 14 is a side elevation view of the goggles of FIG. 12;

FIG. 15 is a front elevation view of the goggles of FIG. 12;

FIG. 16 is a rear elevation view of the goggles of FIG. 12;

FIG. 17 is a perspective view of the helmet of FIG. 9 having the movable jaw in the closed position;

FIG. 18 is a side view of the chin bar with an hinge element;

FIG. 19 is a perspective view of the movable jaw shield lower portion;

FIG. 20 is a perspective close-up view of the movable jaw shield;

FIG. 21 is perspective view of a portion of the helmet outer shell;

FIG. 22 is a perspective view of the helmet outer shell underneath an optional sun visor;

FIG. 23 is a side elevational view of another embodiment of a movable jaw shield; and

FIG. 24 is a rear perspective view of goggles according to one embodiment.

### DETAILED DESCRIPTION OF A PREFERRED EMBODIMENT

A novel helmet having a movable jaw shield will be described hereinafter. Although the invention is described in terms of specific illustrative embodiments, it is to be understood that the embodiments described herein are by way of example only and that the scope of the invention is not intended to be limited thereby.

The use of the expression “MX helmet” throughout this disclosure should be understood as a motocross and/or off-road helmet typically having elongated chin and preferably visor portions, a chin bar, and partially open face to give the rider extra protection while wearing goggles and to allow the unhindered flow of air during the physical exertion of this type of riding. The visor is to allow the rider to dip his or her head and provide further protection from flying debris during off road riding. It will also keep the sun out of the eyes of the rider during jumps. Though referring to motocross and off-roads, such helmet may be used for recreational vehicle such as ATV, UTV, snowmobile and the likes.

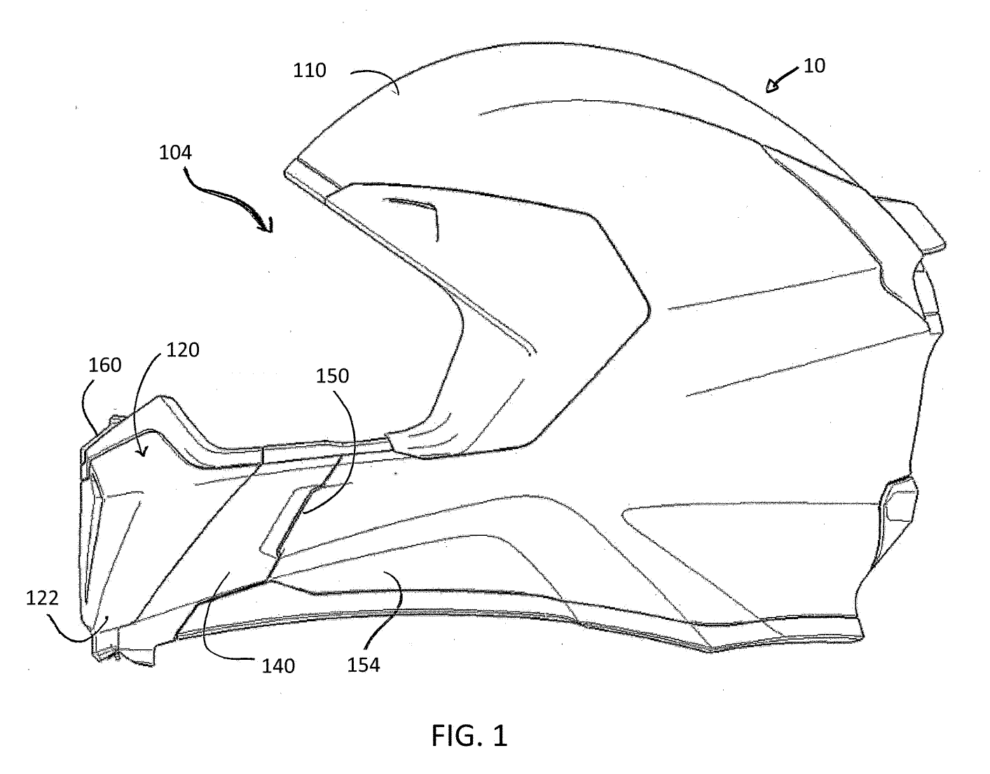

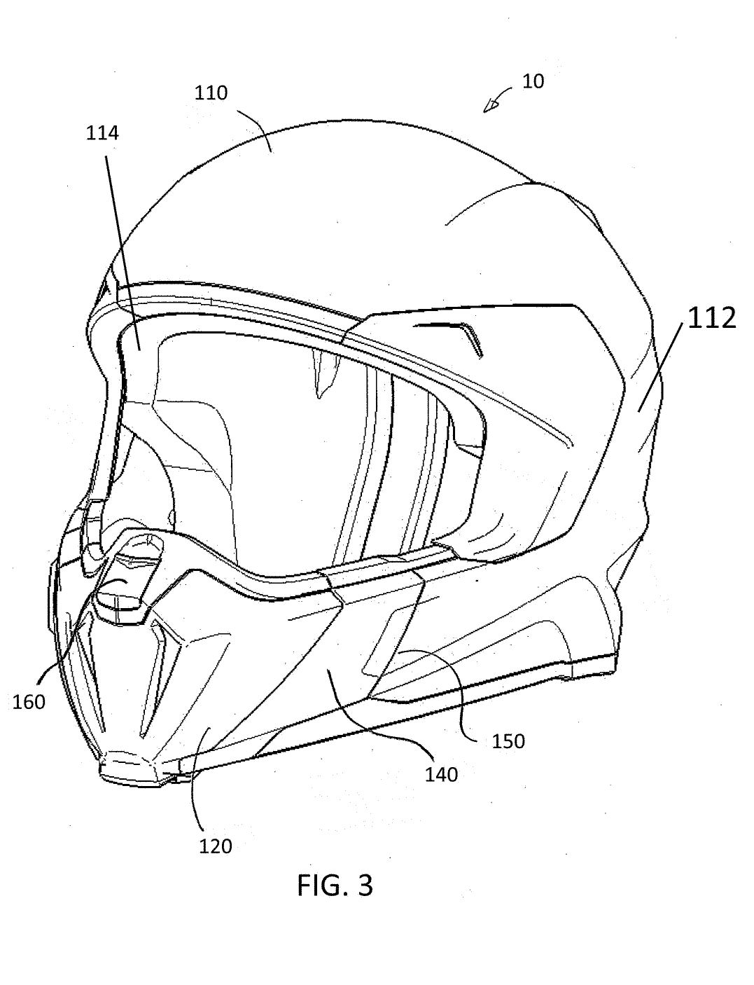

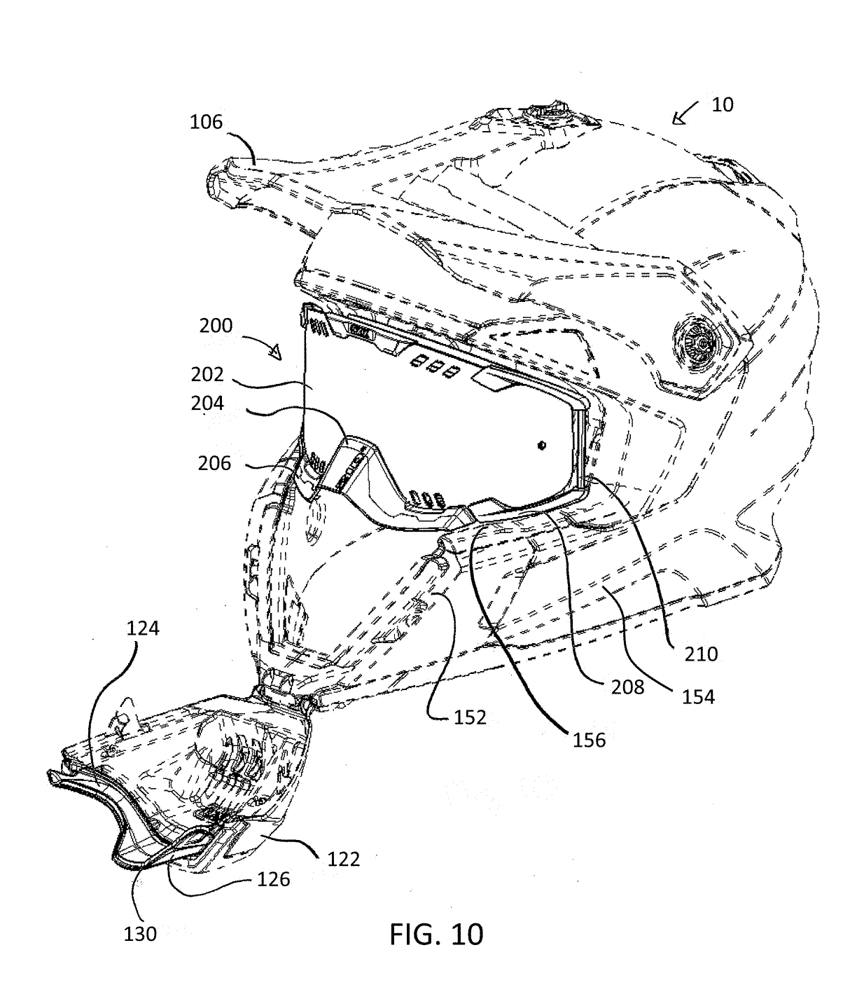

Now referring to FIGS. 1-3, according to one embodiment, the helmet 10 comprises of a head portion 110, elongated chin portions 140 forming a chin bar 154 and a partially open face 104. In addition, the helmet 10 may comprise visor portions 106 (see FIG. 10). The chin bar 154 further comprises a jaw shield 120. The helmet 10 may further comprise a belt or strap (not shown) to secure the helmet 10 on the user's head. The helmet 10 according to embodiments comprises a movable/openable jaw shield 120. As illustrated, the jaw shield 120 is typically mating with the head portion 110 along its chin portion elongated side edges 140 thus creating an additional open space to the partially open face portion 104. The helmet 10 according to embodiments shown in FIGS. 1-3 is an MX-type helmet, a helmet deprived of face shield or fixed transparent visor. The visor/eye shield in MX helmets is generally replaced by suitable goggles 200 (see FIG. 10).

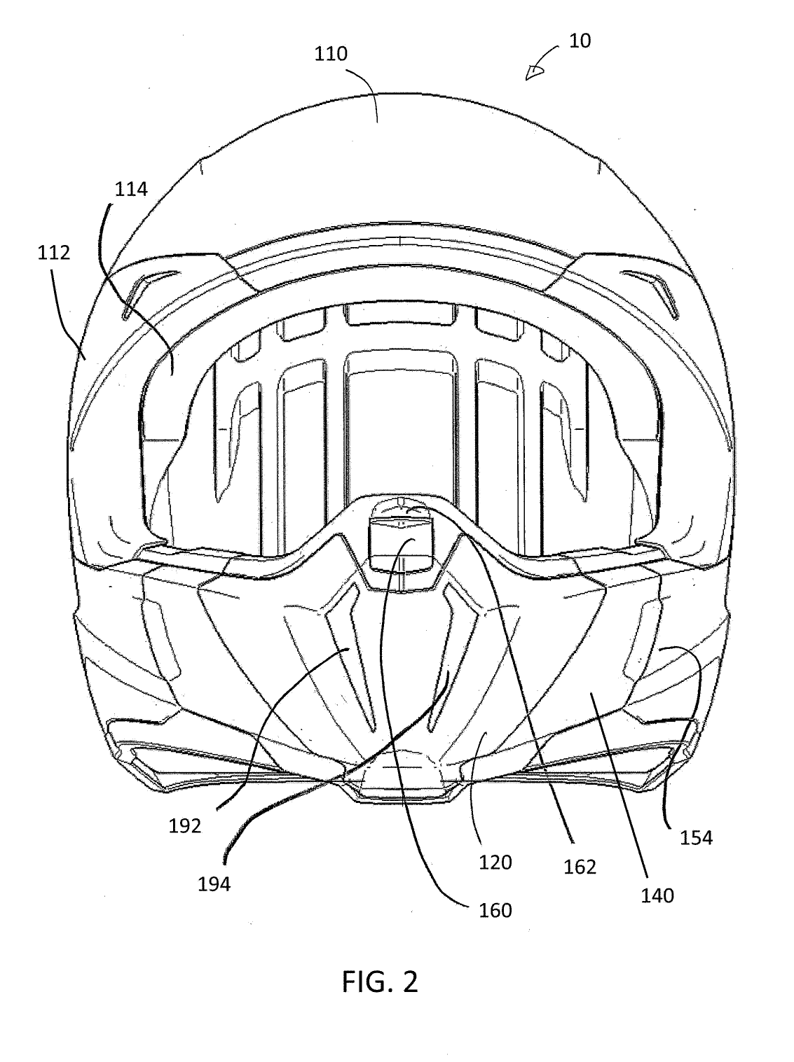

According to embodiments, further referring to FIG. 1-4, the movable jaw shield 120 comprises a shield portion 122, a hinge 190 for pivotally mounting the shield portion 122 to the chin bar elongated side edges 140, releasable securing members 182 and a release mechanism 160. The releasable securing members 182, such as latch type securing members 182, are controlled by the release mechanism 160. According to the embodiment shown in FIGS. 1-4, the release mechanism is a lever 160 actuating securing latches 182 on either side of the inner face of shield portion 122. The actuation of the lever 160 releases the latches 182 from the chin bar side edges 140 attachment openings 170, 172 to allow outward movement of the movable jaw portion 120. As such, the actuation of the lever 160 by the user permits the opening and removal of the movable jaw portion 120 as shown by FIG. 2. The movable jaw shield 120 also comprises a lever cavity 162 located behind the lever 160 to ease opening of the jaw portion 120 when the user is wearing some protection gear or insulating wear accessories. As such the lever cavity is used to facilitate access to the lever for user which may be wearing cumbersome hand gear.

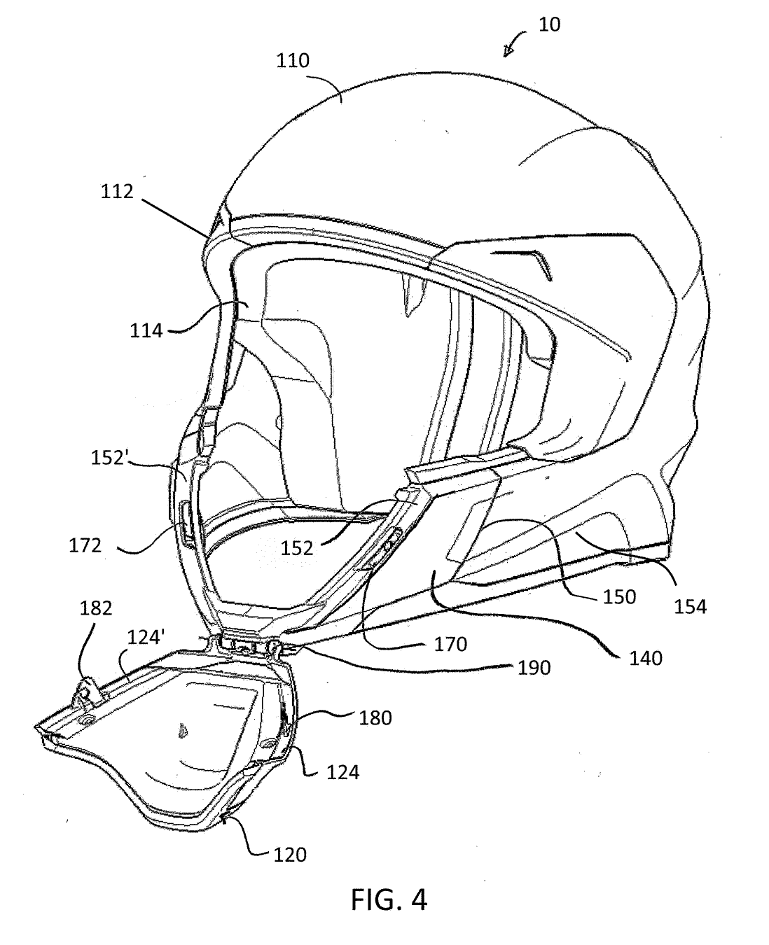

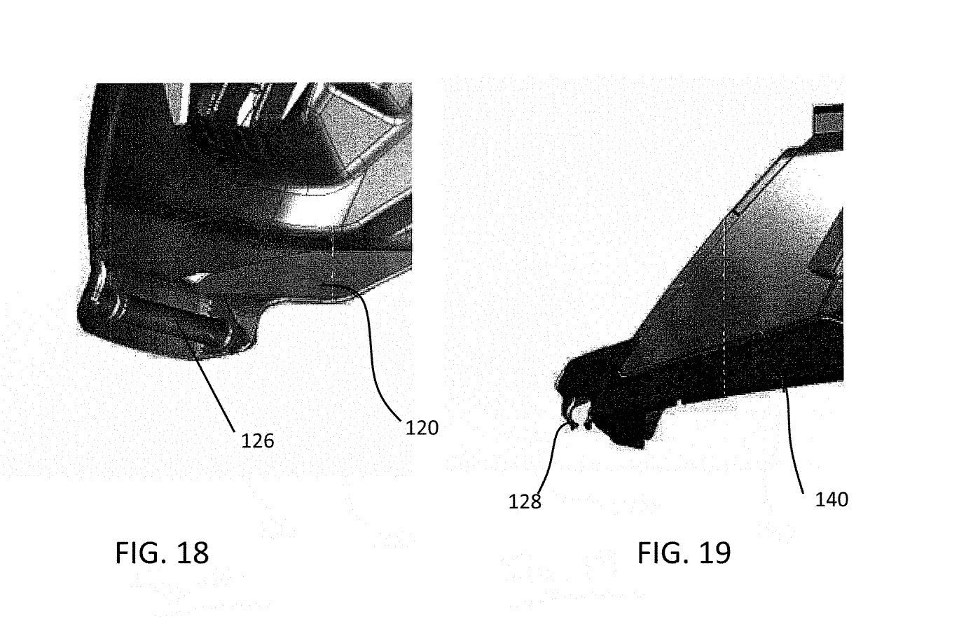

Now referring to FIG. 4, according to embodiments, the movable jaw shield 120 is pivotally mounted to the chin bar 154, preferably through hinges 190 allowing the outward movement of the jaw shield 120 to favor ease of opening while the helmet 10 is in use. In the present embodiment, the movable jaw shield portion 120 is reversibly mounted to the lower portion of the chin bar 154 via hinges 190. Understandably, the chin bar 154 has a reduced height at the front portion of the helmet 10 leaving some open space for the movable jaw shield 120. As shown in FIGS. 18-19, the movable jaw shield 120 lower portion comprises a hinge male 126 element configured to be received in the corresponding female element, a C-shaped receiving element 128 mounted on the front lowermost portion of the chin bar elongated side edges 140. Understandably, though the present configuration is preferred, other embodiments could as well have hinges 190 with the male and female portions inverted. Likewise, though the hinge 190 mechanism used for pivotally moving the movable jaw portion is preferred, other suitable securing mechanisms may be used so long as the securing mechanism allow the removal of the jaw shield portion. Referring back to FIG. 4, the movable jaw shield 120 in accordance with the present invention comprises a hinge 190 mechanism may be reversibly mounted on the helmet chin bar 154. The movable jaw shield 120 can be opened and closed by pivoting at the hinge 190 in the lower front portion thereof. The mechanism also allows removal of the jaw shield 120 by unmounting the male latch element from the female element. Accordingly, the movable jaw shield may be detached entirely from the helmet for replacement or customization. Additionally, the movable jaw shield 120 may be replaced by another jaw shield depending on the kind of use and weather conditions. For instances, some movable jaw shields comprise ventilation controls. As such different ventilation control and/or air filter may be desired to optimise the helmet breath box behavior. Furthermore, due to the outer shell 112 configuration, when the movable jaw shield is removed, the helmet 10 keeps its structural integrity. The main shell 112 (also referred as outer shell) is preferably made of fiberglass. In addition, the inner side 114 or EPS side (rigid foam) preferably substantially covers the interior front for added protection. Other embodiments may comprise hinges 190 having the male 126 and female 128 portions (See FIGS. 18 and 19) fixedly pivotally mounted resulting in a movable jaw shield that is not replaceable or not easily replaceable.

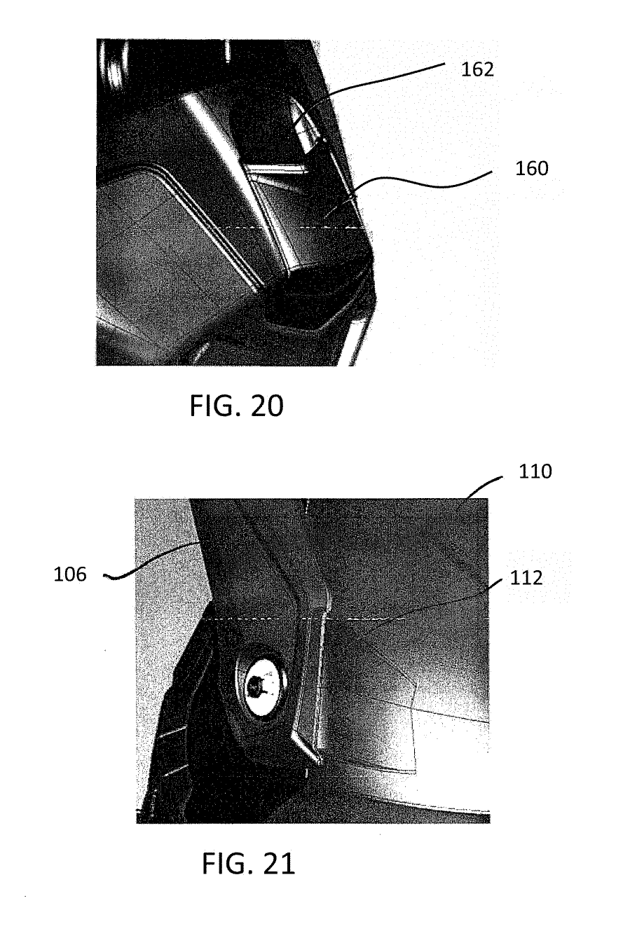

Now referring to FIGS. 1 to 3, the lever 160, according to the present embodiment, is designed with two materials (through a molding process). The outer part of the lever 160, the visible portion, is made from a plastic material, whereas the inner part, the inside portion (not shown) may be made of rubber. This lever 160 configuration allows improved sealing of the area between the lever 160 and the part that receives it, the opening of the shield portion 122. In addition, the lever 160 is positioned on the movable jaw shield 120 in a way that favors the natural movement of the user's hand by inserting of a finger in the lever opening 162 and pulling outwardly to release the movable jaw shield 120 from its mounted/closed position (also see FIG. 20). The movement of the lever 160 also preferably seals the opening of the jaw shield portion 120, thus preventing unwanted environmental element from entering there through.

Now referring back to FIG. 4 the helmet 10 is shown having the movable jaw shield 120 in open configuration. As illustrated, the movable jaw shield 120 hangs from lower front portion of the chin bar 154 according to the pivot elements 190 located on lower front portion of the chin bar 154. Accordingly, in the open configuration, the movable jaw shield 120 has its upper edge portion (the portion generally about the nose and face of the user when in the closed configuration) located lower than the bottom edge of the chin bar 154. Thereby, the space in front of the place where the mouth of a person wearing the helmet 10 would be freed of obstacles, so the sound of their voice would more easily travel to another person. As such, the helmet 10 generally improves communication between the user and another person. The user may thus selectively open the jaw shield 120 to discuss for instance with another user without the need for removing the helmet 10 as a whole.

Still referring to FIG. 4, according to an embodiment, the outer layer of the front side edges 152, 152′ of the head portion 110 of the helmet 10 may be mating with the inner layer 114 of the jaw shield 120. That depends in part in the design of the inner layer(s) such as the thickness of the inner layers, or the presence of an inner layer in both components.

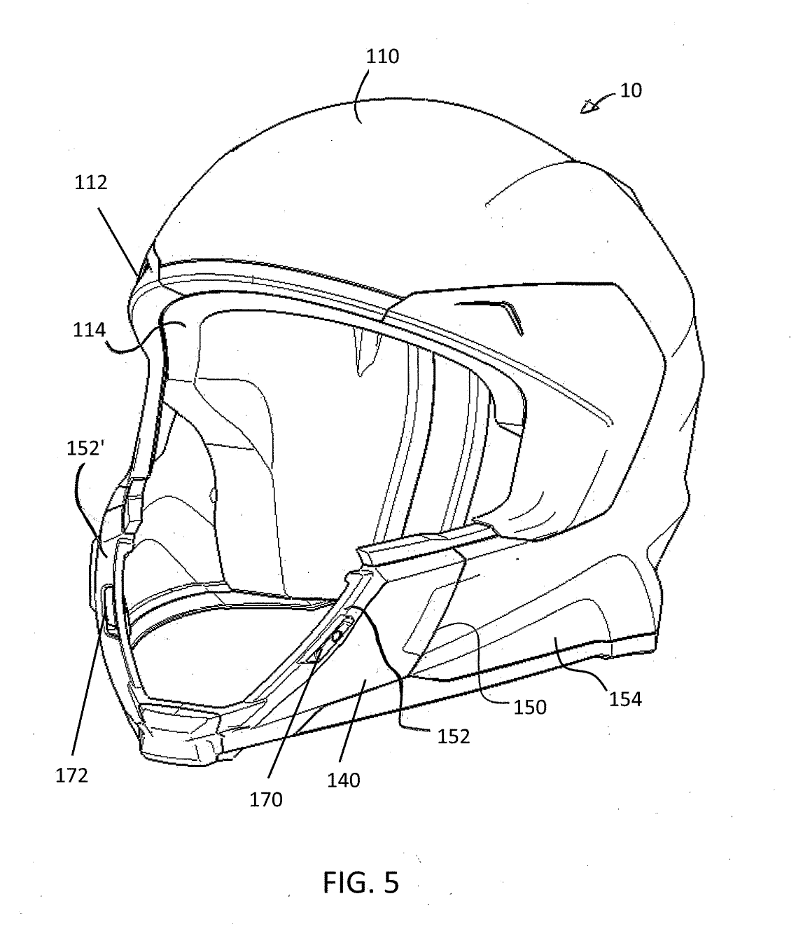

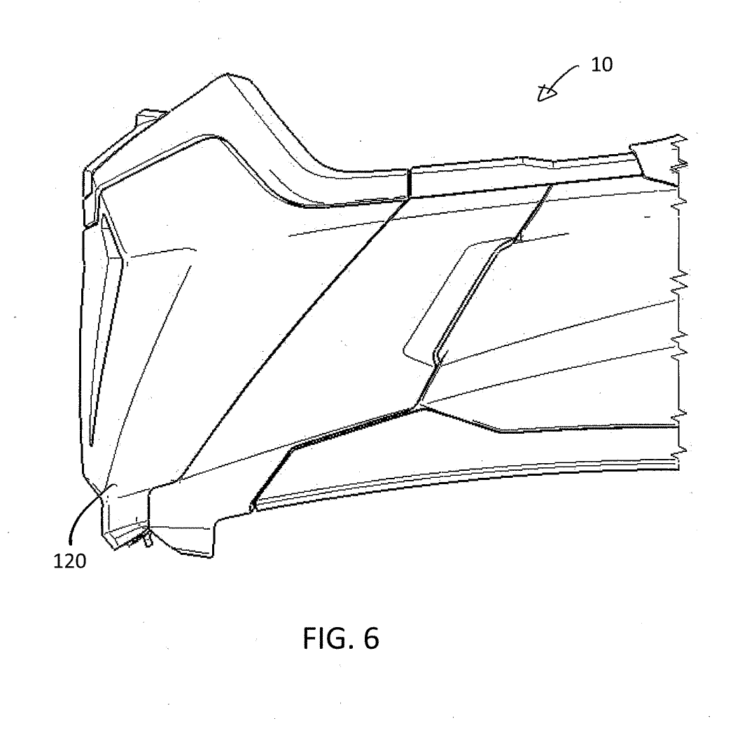

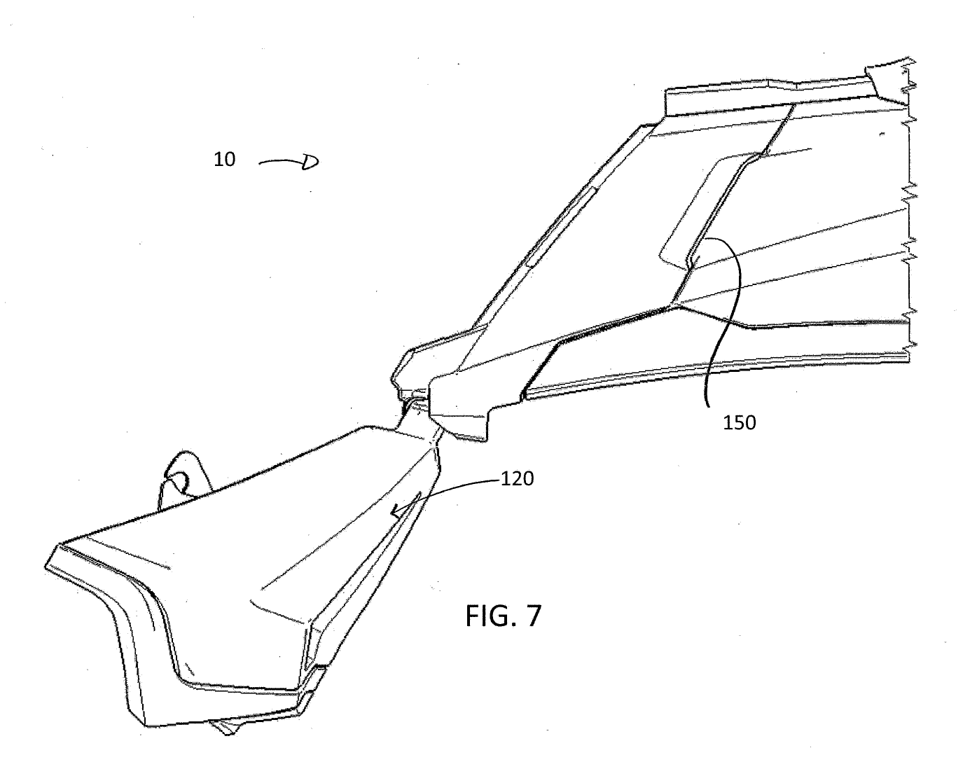

In FIG. 5, the helmet 10 is shown without the movable jaw shield 120. According to one embodiment, the helmet 10 is made of a continuous structure thus retaining the structural strength and integrity of head portion even in the absence of the movable jaw shield 120. According to an embodiment, the helmet 10 the head portion 110 is generally composed of two layers: a head shell or head portion 110 forming a rigid structure protecting the head of the user, and an intermediate jaw portions 140 having the securing openings 170, 172 located thereto for retaining the jaw shield 120 in the closed/mounted position. Accordingly, when the jaw shield 120 is mating with the head portion 110, the inner sides of the jaw shield 120 are substantially in contact with the forward sides of the intermediate jaw portion 152, 152′ of the chin bar 154.

Referring to FIGS. 2-5, according to one embodiment, the helmet further comprises a robust outer shell (providing protection) 112 an inner layer 114 preferably made of compressible material for comfort and additional security. The jaw shield may also comprise a similar set of an outer shell and an inner layer. So when the helmet is in a closed configuration, the outer shells of the head portion and the jaw shield are substantially mating and the inner layers are compressively mating so that the mating of the two components generates substantially airtight surfaces for maximum comfort. In cases where the helmet 10 has with two inner layers designed to mate, when the compressible material of the inner layer 114 enters in contact with the inner layer of the jaw shield 120, the compressible material of the two layers are slightly compressed therefore creating two airtight surfaces, one on each side of the helmet 10 according to that mating.

When in the closed configuration, the front-edge surfaces 152, 152′ of the head portion chin bar 154 and the back-edge surfaces 124, 124′ of the jaw shield 120 are mating. When in the opened configuration, the front-edge surface of the head portion 110 and the back-edge surface of the jaw shield 120 are free of any contact.

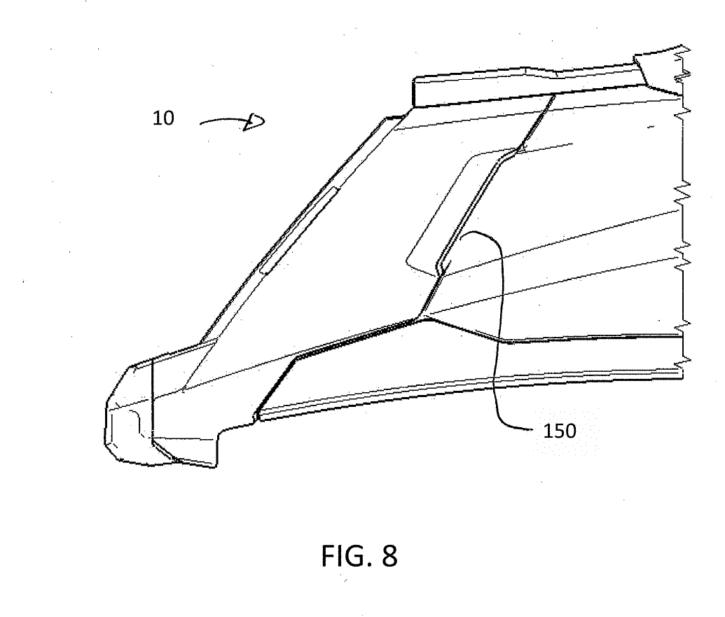

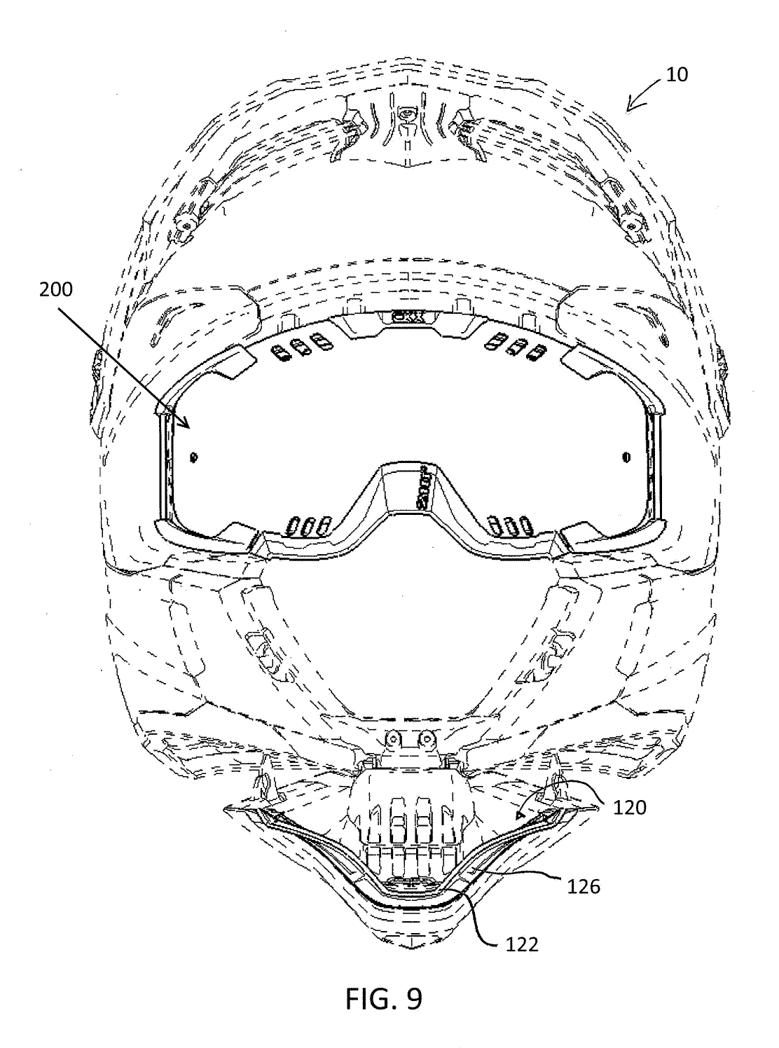

According to another embodiment, a helmet is part of a helmet assembly comprising a helmet and suitably configured goggles 200 (See FIG. 9) for matingly mounting to the partially open face portion 104 (See FIG. 1). The helmet, according to this embodiment, preferably has a movable jaw shield configured to mate with mounted goggles 200. Therefore, according to this embodiment, the goggles 200 and helmet may both be designed to optimise air flow and minimise air entry within the helmet head portion. In addition, according to embodiments, the helmet and goggles 200 are configured to match one another by having complementary features allowing such optimised air flow.

Detailed embodiments of goggles 200, for example, sports goggles and snow and sand goggles, are disclosed herein, however, it is to be understood that the disclosed embodiments are merely exemplary of the goggles 200, which may be embodied in various forms so long as they are compatible with the movable jaw shield helmet 10. Therefore, specific functional details disclosed herein are not to be interpreted as limiting, but merely as a basis for the claims and as a representative basis for teaching one skilled in the art to variously employ the present disclosure.

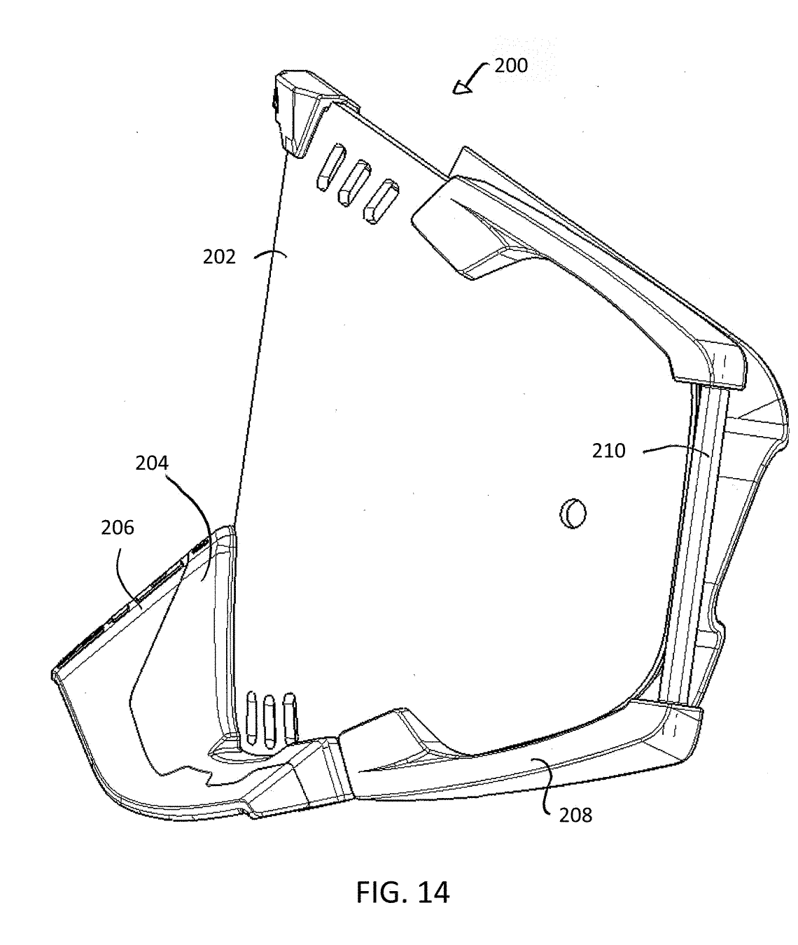

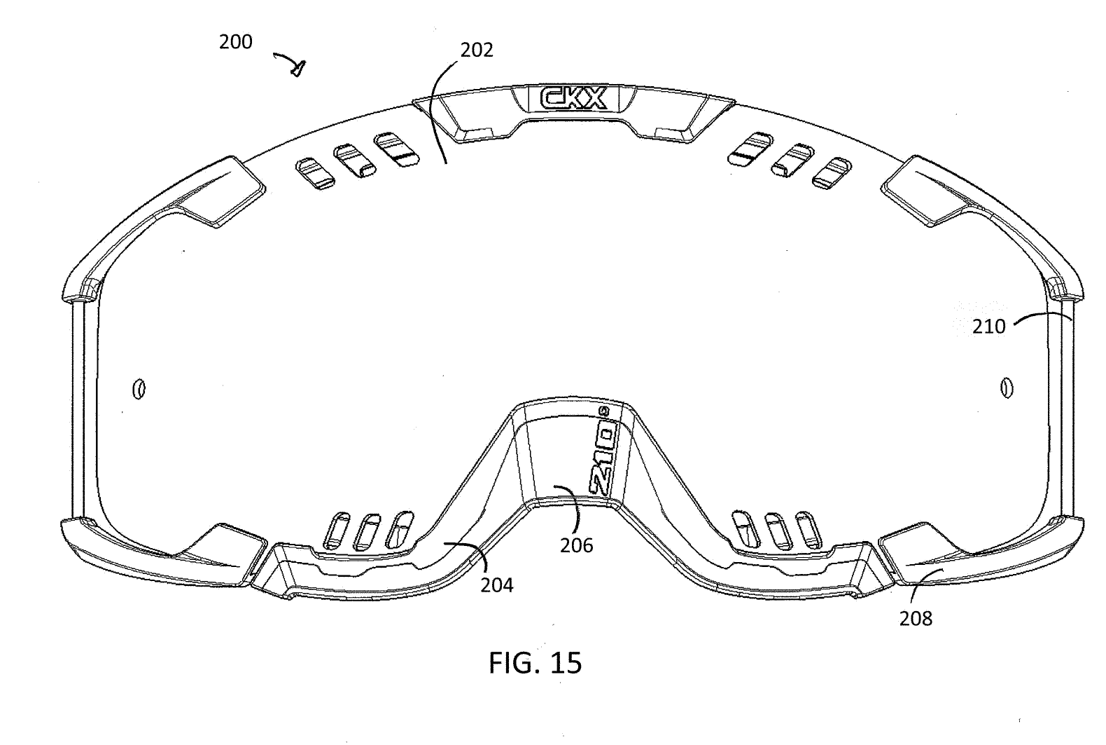

In illustrative embodiments, referring to FIGS. 9-17, goggles 200 compatible with the movable jaw shield helmet 10 that may be used for skiing, snowboarding, motocross, snowmobiling or any other sports where eye protection and visibility is important are disclosed herein. The goggles 200 provide an improved fit with the helmet as compared to other goggles 200 and at least reduce the amount of air generally entering between the goggles lower frame and the breath guard of the movable jaw shield. Thus, the goggles 200 which are compatible with the movable jaw shield helmet 10 have the ability to provide a greater level of comfort in that a wearer or user is less prone to the unwanted chilling effect of air entering between the helmet chin bar and the goggles lower frame. Additional embodiments are related to goggles that are easy to use and allow improved compatibility between the movable jaw shield helmet breath guard and the extended goggle lower frame portion.

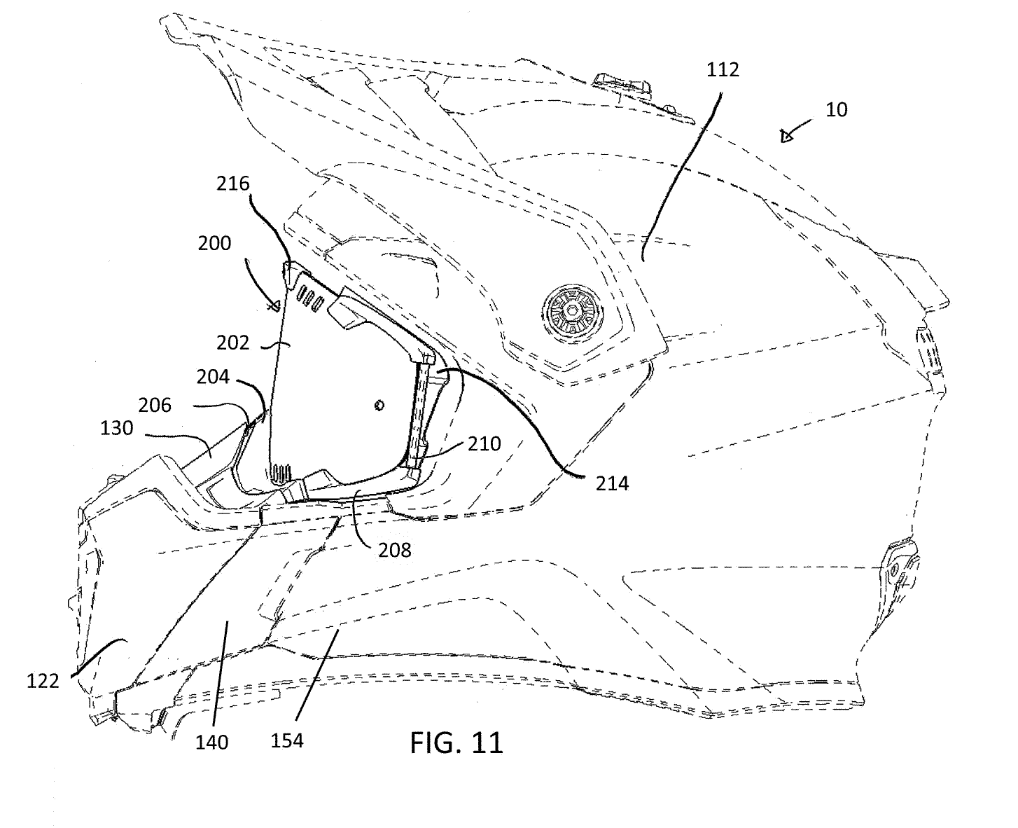

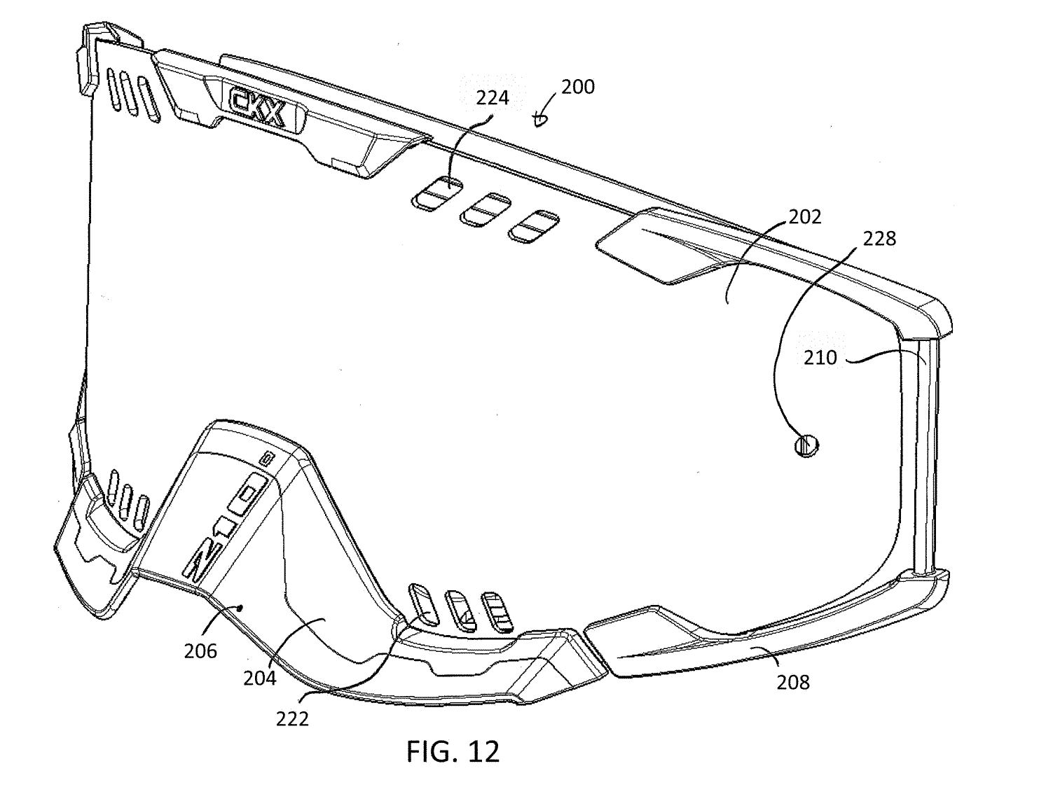

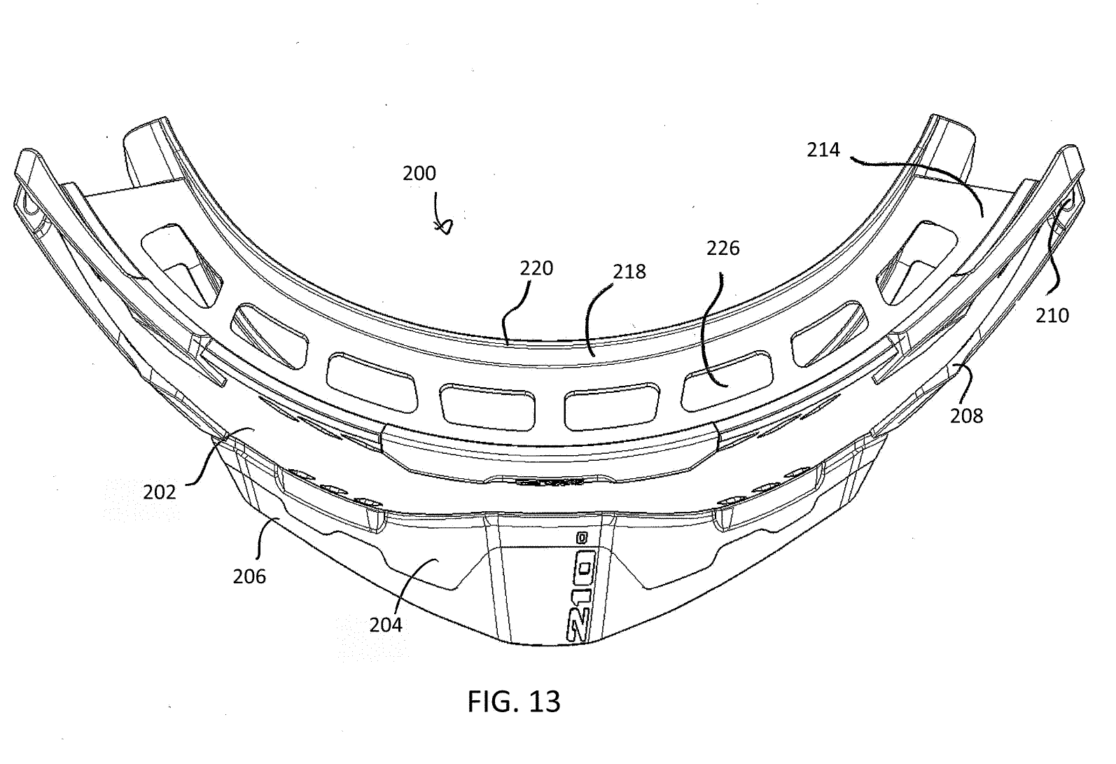

As illustrated in FIG. 11, the goggles 200 comprise a lens assembly 202 adapted to be coupled, optionally in a movable manner, to a frame 214 associated with a goggles assembly 200. The frame 214 comprises an upper portion 216, a lower front portion 204, an extended front portion 206, lower side portions 208, a strap retaining portion 210 and a support portion 218 (see FIG. 13) typically retaining a foam layer 220 to improve user comfort when wearing the goggles. As shown by FIG. 13, the goggles frame 214 may comprise one or more frame venting apertures 226 on one or more of a top portion and a bottom portion of the goggles frame 214, typically behind the lens. Referring now to FIG. 12, the lens 202 may also comprise one or more venting apertures 222, 224, 228 which extend from the outer surface to the inner surface and may be disposed or located in various locations on the lens 202. In the illustrative embodiment of FIG. 12, the one or more venting apertures 222, 224 are located in a lower portion and upper portions of the lens 202.

According to one embodiment, now referring to FIGS. 10 and 11, the goggles are configured to be mounted on the helmet 10. As such, in the present embodiment, the jaw shield upper portion comprises a breath guard 130 designed to mate with the goggles 200 extended front lower portion 206 thus increasing the aerodynamics of the helmet 10 and preferably reducing the amount of incoming environmental elements between the helmet and goggles 200. Accordingly, when the movable jaw shield 120 is in a closed position (FIG. 11), the upper outer surface of the extended front lower portion 206 mate with the inner surface 124 of the breath guard 130. The breath guard 130 and goggles extended front lower portion 206 are preferably made of a flexible polymeric material such as rubber. Therefore, the mating of the breath guard 130, also referred as nose guard or nose shield, with the extended front lower portion 206 results in a preferably weather proof connection effectively preventing environmental elements or debris from entering the inside of the helmet 10 and effecting the comfort of the user. According to one embodiment, the chin bar upper surface 156 is also configured to mate with the goggles lower side portions 208 also preferably reducing the amount of environmental element generally entering the helmet 10 between the chin bar 154 and goggles 200.

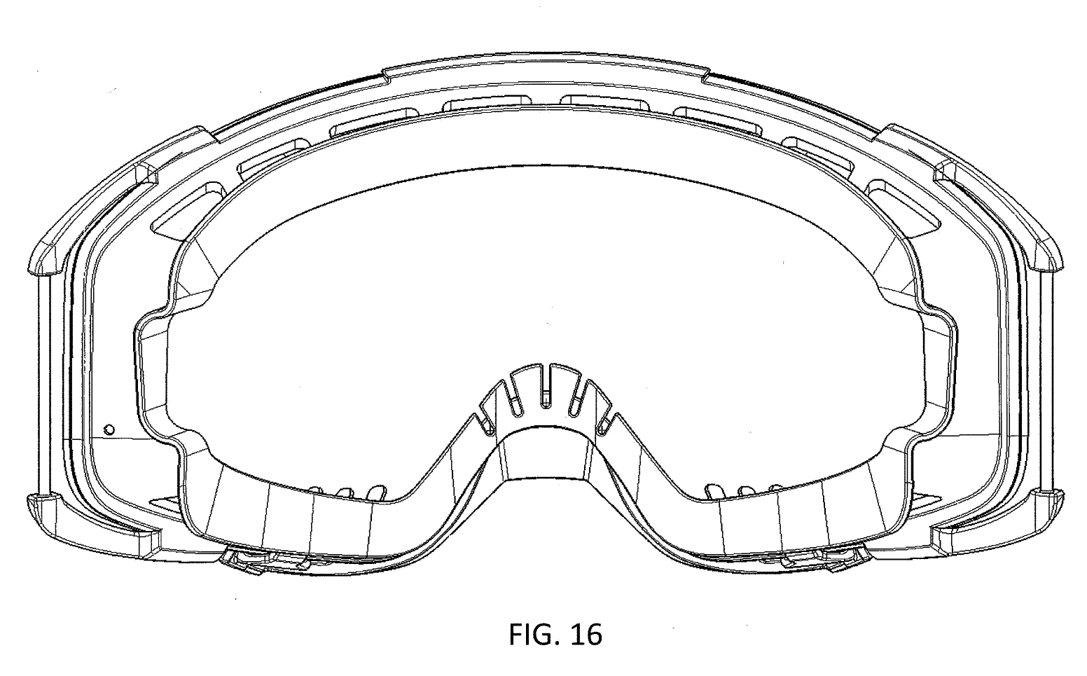

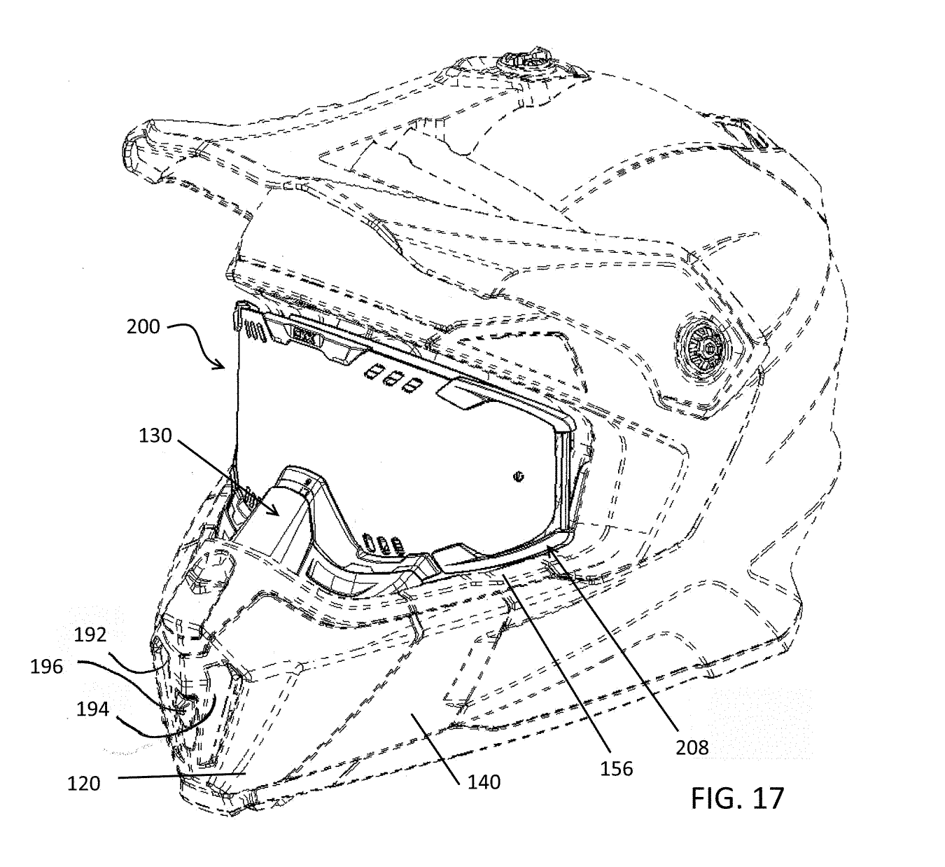

According to one embodiment, now referring to FIGS. 2 and 17, the movable jaw shield 120 further comprises a ventilation control 194 enabling the user to manage the amount of air allowed to enter and exit through the movable jaw shield 120 air intakes 192, 194. According to this embodiment, the MX helmet 10 with movable jaw shield 120 is capable of behaving like full face helmet due to the creation of a controlled breath box area between the movable jaw shield 120, the forward chin bar elongated sides 140 and the user's face. The mating of the goggles 200 with the breath guard 130 and upper sides 156 of the chin bar 154 with the lower sides 208 of the goggles 200 create the breath box and generally allow the user an increased level of control of the incoming air.

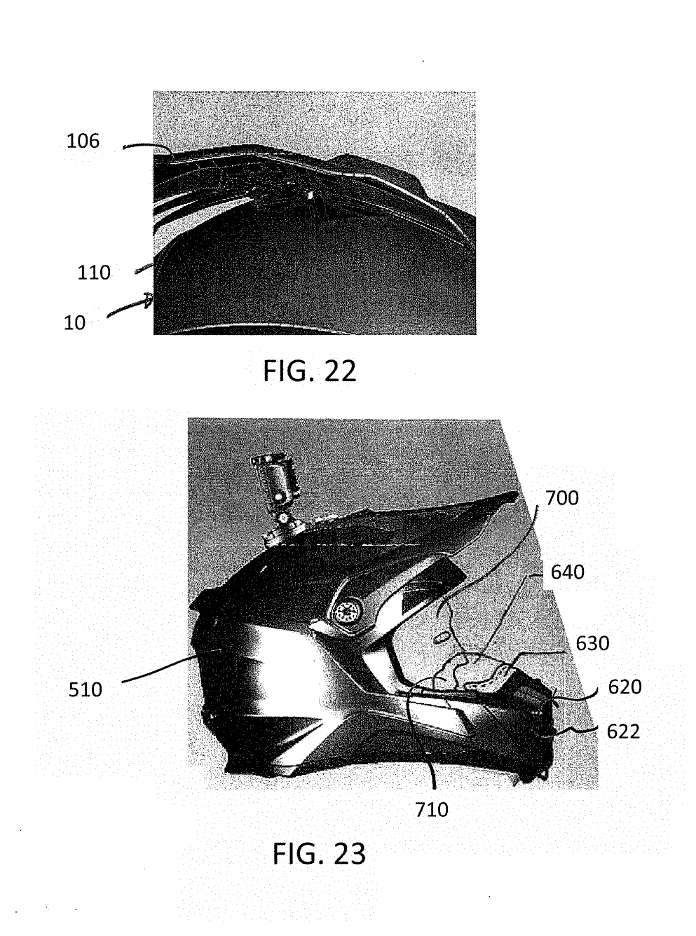

According to one embodiment, now referring to FIGS. 21-22, the helmet 10 further comprises an air diffuser underneath the helmet optional sun visor 106 and a clearance of the shell 112 relative to the optional sun visor 106 to reduce the wind resistance and improve the helmet 10 aerodynamics.

According to another embodiment, now referring to FIG. 23, the movable jaw shield 620 comprises a breath guard 630 designed to be in contact with the upper portion of the nose and portions of the face below the eyes when the movable jaw shield 620 is in closed position (FIG. 23). The movable jaw shield 620 comprises a jaw shield portion 622, a rigid guard 630 and a flexible breath guard 640. The rigid guard 630 is generally mounted on the inner top portion of the movable jaw shield 620 as to project inwardly toward the face of the user 700. The flexible breath guard 640 according to this embodiment may be made from textile or polymeric material such as rubbers and is generally mounted on the rigid guard 630 extending further inwardly to mate with the user's face. The type of material used for the flexible breath guard 640 will vary depending on the intended use of the helmet 510. As such, rubber materials may be preferred for use in cold weather conditions, whereas textile may prevent over sweating of the user's face 700 in warmer weather conditions. The flexible breath guard 640 according to this embodiment is configured to mate with the under surfaces of the goggles frame mounted on top thereof (not shown). Consequently, when the user puts the goggles on, the undersurface of the goggles frame pressures the flexible breath guard 640 on the user nose and face 710 thus potentially sealingly mounting the goggles thereto. Notably, the helmet 510 flexible breath guard 640 or breath guard assembly and goggles help reduce the unwanted intrusion in the helmet breath box of unwanted environmental element such as air, sand, dirt, snow or any other material potentially affecting the comfort and performance of the user. According to this embodiment, the helmet with movable jaw shield 510 may be used as a conventional MX helmet 510 that is the user puts on the helmet 510 and then closes the jaw shield if not closed and puts on the goggles afterwards. Depending on the flexibility of the flexible breath guard 640, the user may be able to open the movable jaw shield 620 while the goggles are on. In such cases the user must carefully insert the flexible breath guard 640 underneath the goggles upon closing the movable jaw shield 620.

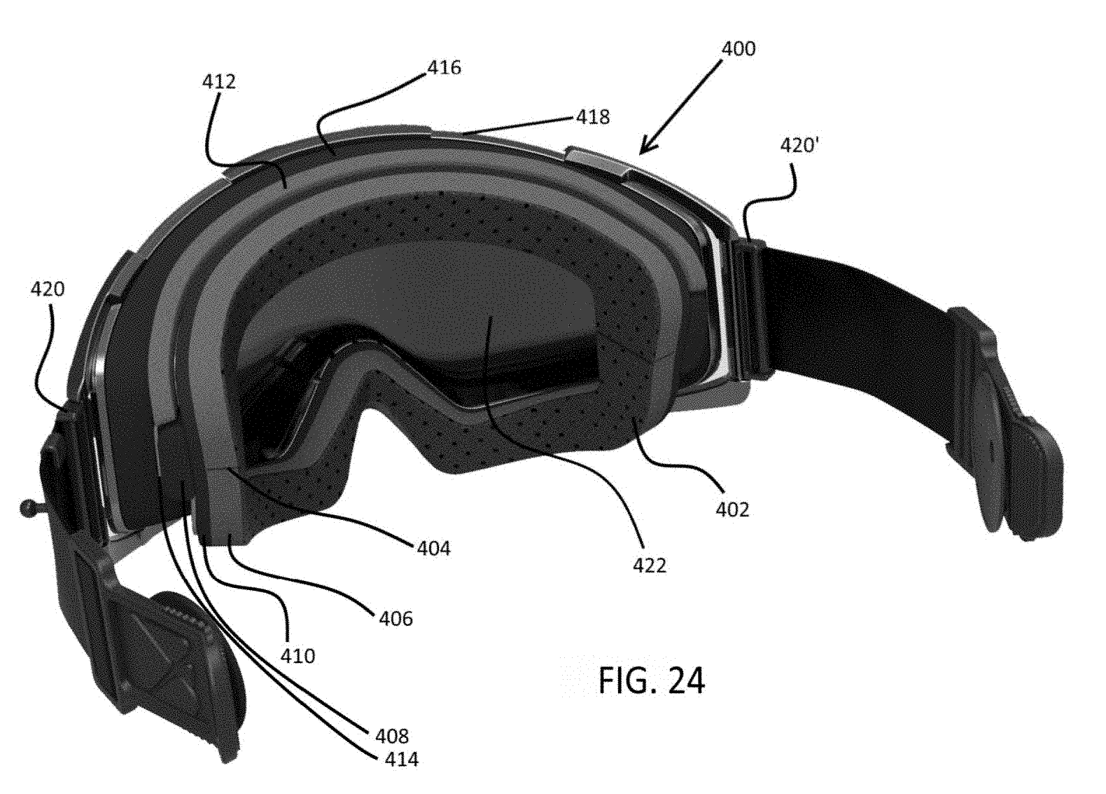

According to another embodiment, now referring to FIG. 24, the goggles 400 compatible with the removable jaw shield helmet 10 comprises goggles frame 418, an intermediate layer 416, a top seal foam layer 412, a closed cell support foam layer 410, a foam contour 406 and a foam dressing fabric 402.

The goggle frame 418 has the intermediate layer 416 mounted thereto. The intermediate layer 416 may be made from open cell foam, from closed cell foam or from a mix of both open and closed cell foam to allow the goggles to be breathable. As such, the intermediate layer is mounted on the goggles 400 frame 418. The frame 418 surrounds the goggles shield 422 and the goggles attachment elements 420, 420′. The top seal foam layer 412 is generally mounted on upper portion of the intermediate layer 416 or directly on the frame 418 which extends underneath the intermediate layer 416. As such, the top seal foam layer is designed to be in contact with the top portion of the helmet eye port (also the upper surface of the eye port). The top seal foam layer 412 is additionally preferably spaced apart from the goggles shield 422. As such, the top seal foam layer 412 is generally the first line of defense against cold wind infiltration. The top seal foam layer 412 is preferably made of closed cell foam.

In addition, to the top seal foam layer 412, the goggles 400 according to this embodiment have a closed cell support foam layer 410 mounted on the goggles frame 418 designed for improving the goggles support. As such, according to some embodiment, the frame 418 may extend upwardly between the top seal foam layer 412 and the closed cell support foam layer 410. The closed cell support foam layer 410 is the second line of defense against cold infiltration. The closed cell support foam layer 410 also has a foam contour layer 406 mounted thereon.

The foam contour layer 406 generally provide added comfort and insulation from the other portions of the goggles. As such, the foam contour 406 generally insulate the driver from the heat and cold arising from the environmental element encountered by the user and as a result of the practiced sport or activity. The foam contour layer 406 may additionally further comprise a foam dressing fabric 402, such as perforated antimicrobial and/or hypoallergenic fabric to enter into contact with the user's head when wearing the goggles 400. The foam contour layer 406 may further comprise a gash or a cut 404 designed to accommodate user eyewear (not shown). User eyewear are generally prescription eyewear but may as well be other types of eyewear. The foam contour layer 406 gash 404 is built to allow user eyewear stem (not shown) to be inserted in the foam contour 406. The foam contour 406 thus moves aside and allows eyewear stem to be inserted in the gash 404 thus at least mitigating the pressure exerted on the eyewear stem in the absence of such feature. In addition, the gash 404 allows an improved comfort felt by the user as the gash 404 permits the foam contour layer 406 and foam dressing fabric 402 to be closer to the user's face as compared with goggles without such gash 404 when wearing eyewear. The gash 404 also allows user without eyewear to use the goggles without any inconvenience. As such, the goggles 404 are usable by users with and without eyewear.

Still referring to FIG. 24, the goggles 404 further comprises eyewear compatible design out bossed foam platform. The out bossed foam platform 408 located in the critical area of the typical location of eyewear stem will enable eyewear stem to locate freely. The out bossed foam platform 408 is generally made of a low density foam block. The out bossed foam platform 408 is designed to seal the area when no eyewear is worn whereas it will compress the area when eyewear are worn. In addition to the out bossed foam platform 408, the goggle frame 418 may move away from the face of the portion in contact with the user's face the combination of layers 410, 406 and 402 at the stem area 414. Accordingly, the featured of the goggles 400 improve the versatility by providing goggles for user wearing eyewear or not.

Through the features of the goggles 400 in FIG. 24 are taught in combination with a removable jaw shield helmet 10, goggles 400 with the features shown in FIG. 24 and described hereinabove could also be used with other MX type helmets. Understandably, the goggles 200 may also comprise the features of the previously described goggles embodiments such as goggles 200 seen in FIGS. 10-17.

According to one embodiment, the method for opening the movable jaw shield comprises a single step of pulling the lever outwardly and downwardly. The pulling of the lever then unsecure the latches and allow the movable jaw shield to follow the outward movement of the hand.

According to one embodiment, the method for removing the jaw shield of the helmet having a movable jaw shield comprises the steps of:
a. pulling the jaw shield lever downwardly; and
b. pulling the jaw shield outwardly.

The method may further comprise the steps of removing the jaw shield entirely by separating the hinge elements. In addition, the method may further comprise the steps of mounting the hinge elements together and pivoting the jaw shield upwardly and inwardly to secure the jaw shield to the helmet side edges using the jaw shield securing members.

According to one embodiment, the method for mounting the jaw shield comprises the steps of:
a. Mounting the movable jaw shield to the helmet chin bar;
b. Upwardly pivoting the jaw shield for the upper portion of the jaw and breath guard portion to come in contact with the nose and face of the user;
c. Securing the jaw shield to the chin bar side edges; and
d. Mounting the goggles 200, 400 on top of the breath guard.

According to another embodiment, the method of mounting the jaw shield comprises the steps of:
a. Mounting the movable jaw shield to the helmet chin bar;
b. Upwardly pivoting the jaw shield for the upper portion of the jaw and its breath guard portion to come in contact with the goggles 200, 400 of the user; and
c. Securing the jaw shield to the chin bar side edges.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1) A helmet comprising:
a) a head portion; and
b) a jaw shield comprising
i) a fixed portion rigidly connected to the head portion, and having a pair of front edges extending forwardly therefrom when the helmet is worn by a user;
ii) a portion movable from an opened to a closed position, said movable portion having a lower portion and an upper portion, the lower portion being attached to the fixed portion via, at least in part, a hinge comprising a first part connected to the moveable portion and a second part connected to the fixed portion, the second part being selectively engageable with the first part, the upper portion comprising a pair of back-edge surfaces each of which is selectively engageable with one said front edge.

2) The helmet as claimed in claim 1), wherein the moveable portion pivots outwardly and downwardly relative to the fixed portion.

3) The helmet as claimed in claim 2), wherein the moveable portion has an attached position and a detached position.

4) The helmet as claimed in claim 3), wherein the helmet comprises a latch mechanism adapted to engage the fixed portion when the moveable portion is in the attached position.

5) The helmet as claimed in claim 1), wherein one of the first and second parts comprises a C-shaped clip extending between free ends of the fixed portion and wherein the other one of the first and second parts comprises a pin that is engageable with the C-shaped clip such that the pin and C-shaped clip being rotatable relative to each other when engaged.

6) The helmet as claimed in claim 4), wherein the latch mechanism comprises at least one hook disposed on one of first and second lateral sides of the moveable portion.

7) The helmet as claimed in claim 6), wherein the latch mechanism comprises at least one catch plate disposed on one of first and second lateral side edges of the fixed portion.

8) The helmet as claimed in claim 7), wherein the at least one hook selectively engages the at least one catch plate when the moveable portion is in the attached position.

9) The helmet as claimed in claim 1), wherein the moveable portion is completely removable from the fixed portion.

10) A helmet assembly comprising:
a) goggles; and
b) a helmet comprising
i) a head portion; and
ii) a jaw shield comprising
a fixed portion rigidly connected to the head portion, and having a pair of front edges extending forwardly therefrom when the helmet is worn by a user;
a portion movable from an opened to a closed position, said movable portion having a lower portion and an upper portion, the lower portion being attached to the fixed portion via, at least in part, a hinge comprising a first part connected to the moveable portion and a second part connected to the fixed portion, the second part being selectively engageable with the first part, the upper portion comprising a pair of back-edge surfaces each of which is selectively engageable with one said front edge and being in contact with the goggles when in the closed position.

11) The helmet assembly as claimed in claim 10), wherein the moveable portion is configured to mate with a lower portion of the goggles.

12) The helmet assembly as claimed in claim 10), wherein the jaw shield is pivotally coupled to the head portion.

13) The helmet assembly as claimed in claim 12), wherein the jaw shield is moveably coupled to the head portion according to two pivot elements, each one situated on a side of the head portion at a lower front edge of the head portion.

14) The helmet assembly as claimed in claim 10), wherein the head portion comprises an outer shell and an inner compressible layer.

15) The helmet assembly as claimed in claim 14), wherein the jaw shield comprises an outer shell and an inner compressible layer.

16) The helmet assembly as claimed in claim 15), wherein the helmet assembly being in the closed configuration and the outer shells of the head portion and of the jaw shield are substantially mating and their inner layers are compressively mating so that the mating of both components generates substantially airtight surfaces for maximum comfort.

17) A helmet assembly comprising:
a) goggles; and
b) a helmet comprising
i) a head portion;
ii) a jaw shield comprising
a fixed portion rigidly connected to the head portion, and having a pair of front edges extending forwardly therefrom when the helmet is worn by a user;
a portion movable from an opened to a closed position, said movable portion having a lower portion and an upper portion, the lower portion being removably attached to the fixed portion via, at least in part, a hinge comprising a first part connected to the moveable portion and a second part connected to the fixed portion, the second part being selectively engageable with the first part, the upper portion comprising a pair of back-edge surfaces each of which is selectively engageable with one said front edge and being in contact with the goggles when a closed position; and
a breath guard.

18) The helmet assembly as claimed in claim 17), wherein the breath guard is preferably made of a flexible polymeric material such as rubber.

19) The helmet assembly as claimed in claim 17), wherein the jaw shield comprises a ventilation control enabling a helmet's user to manage the amount of air allowed to enter and exit through the jaw shield.

20) Helmet goggles comprising:
a) a lens assembly having an outer surface and an inner surface; and
b) a frame comprising:
an upper portion;
a lower front portion;
an extended front portion;
lower side portions;
a strap retaining portion; and
a support portion having a foam layer to improve user comfort when wearing the goggles.

21) The helmet goggles as claimed in claim 20), wherein the frame comprises at least one venting aperture extending from the outer surface to the inner surface of the lens assembly.

22) The helmet goggles as claimed in claim 20), wherein the helmet goggles the lower front portion of the frame is adapted to mate with a breath guard of the helmet moveable jaw shield to provide an improved air insulation.

23) The helmet goggles as claimed in claim 20), wherein the extended front lower portion is preferably made of a flexible polymeric material such as rubber.

24) The helmet goggles as claimed in claim 22), wherein the lower side portions of goggles is adapted to mate a chin bar upper surface of the helmet to provide an improved air insulation.

25) The helmet goggles as claimed in claim 24), wherein the mating of the goggles with the breath guard of the helmet and with the chin bar of the helmet creates a breath box allowing to a user an increased level of control of the incoming air.

26) The helmet goggles as claimed in claim 20), wherein the helmet goggles comprise an intermediate layer mounted on the frame, the intermediate layer being made from an open cell foam or a closed cell foam or a mix of both open and closed cell foam to allow the goggles to be breathable.

27) The helmet goggles as claimed in claim 20), wherein a top seal foam layer is either mounted on an upper portion of the intermediate layer or directly on a frame portion which extends underneath the intermediate layer.

28) The helmet goggles as claimed in claim 27), wherein the top seal foam layer is configured to be in contact with a top portion of a helmet eye port.

29) The helmet goggles as claimed in claim 27), wherein the top seal foam layer is made of a closed cell foam.

30) The helmet goggles as claimed in claim 20), wherein the helmet goggles comprise a closed cell support foam layer mounted on the frame, the closed cell support foam layer being configured for improving the goggles support.

31) The helmet goggles as claimed in claim 30), wherein a foam counter layer is mounted on the closed cell support foam layer.

32) The helmet goggles as claimed in claim 31), wherein the foam contour layer comprises a foam dressing fabric, such as perforated antimicrobial and/or hypoallergenic fabric to enter into contact with the user's head when wearing the goggles.

33) The helmet goggles as claimed in claim 20), wherein the helmet goggles comprise an out bossed foam platform, the out bossed foam platform being located in a critical area of a typical location of eyewear stem to enable eyewear stem to locate freely.

34) A method for opening a helmet jaw shield comprises the following steps:
pulling a jaw shield lever downwardly to unsecure latches; and
pulling the jaw shield outwardly.

35) A method for mounting a jaw shield to a helmet comprises the following steps:
mounting the jaw shield to a helmet chin bar hinge;
upwardly pivoting the jaw shield until an upper portion of the jaw shield is in contact with the nose and face of the user;
securing the jaw shield to the helmet chin bar side edges; and
mounting helmet goggles on top of the jaw shield.
